(19) United States
(12) Patent Application Publication
(10) Pub. No.: US 2012/0309776 A1
(43) Pub. Date: Dec. 6, 2012

(54) PYRROLO[2,3-D]PYRIMIDINE UREA COMPOUNDS AS JAK INHIBITORS

(75) Inventors: Ashley Fenwick, Richland, MI (US); Timothy Allen Johnson, Richland, MI (US); Graham M. Kyne, Portage, MI (US)

(73) Assignee: Pfitzer Inc.

(21) Appl. No.: 13/576,685

(22) PCT Filed: Jan. 24, 2011
(86) PCT No.: PCT/US11/22216

§ 371 Date: Aug. 2, 2012

Related U.S. Application Data
(60) Provisional application No. 61/301,915, filed on Feb. 5, 2010.

(51) Int. Cl. A61K 31/519; A61P 37/08; A61P 17/00; A61P 19/02; A61P 25/00; A61P 29/00; A61P 11/00; A61P 27/02; C07D 487/04; A61P 17/04

(52) U.S. Cl. 514/265.1; 544/280

(57) ABSTRACT

The present invention provides pyrrolo{2,3-d}pyrimidine compounds, their use as Janus Kinase (JAK) inhibitors, pharmaceutical compositions containing this compounds, and methods for the preparation of these compounds.

### FIELD OF THE INVENTION

Described herein are 7H-pyrrolo[2,3-d]pyrimidin-4-yl)amino]-N-piperidine-1-carboxamide derivatives, their analogues, their use as Janus Kinase (JAK) inhibitors, pharmaceutical compositions containing these compounds, and methods for the preparation of these compounds.

### BACKGROUND OF THE INVENTION

Protein kinases are families of enzymes that catalyze the phosphorylation of specific residues in proteins, broadly classified into tyrosine and serine/threonine kinases. Inappropriate kinase activity, arising from mutation, over-expression, or inappropriate regulation, dys-regulation or de-regulation, as well as over- or under-production of growth factors or cytokines has been implicated in many diseases, including but not limited to cancer, cardiovascular diseases, allergies, asthma and other respiratory diseases, autoimmune diseases, inflammatory diseases, bone diseases, metabolic disorders, and neurological and neurodegenerative disorders such as Alzheimer's disease. Inappropriate kinase activity triggers a variety of biological cellular responses relating to cell growth, cell differentiation, survival, apoptosis, mitogenesis, cell cycle control, and cell mobility implicated in the aforementioned and related diseases.

Thus, protein kinases have emerged as an important class of enzymes as targets for therapeutic intervention. In particular, the JAK family of cellular protein tyrosine kinases (JAK-1, JAK-2, JAK-3, and Tyk-2) play a central role in cytokine signaling (Kisseleva et al, Gene, 2002, 285, 1; Yamaoka et al., Genome Biology, 2004, 5, 253). Upon binding to their receptors, cytokines activate JAK which then phosphorylate the cytokine receptor, thereby creating docking sites for signaling molecules, notably, members of the signal transducer and activator of transcription (STAT) family that ultimately lead to gene expression. Numerous cytokines are known to activate the JAK family.

Accordingly, there remains a need for alternative compounds that effectively inhibit JAK enzymes, including JAK-1, JAK-2, JAK-3, and/or Tyk-2.

### SUMMARY OF THE INVENTION

The present invention provides a compound of formula I:

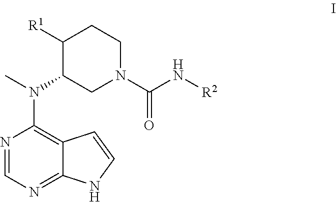

or a pharmaceutically acceptable salt thereof wherein R¹ is H or —C₁₋₄alkyl; and R² is a thiadiazole group optionally substituted with —OH or —OCH₃.

Specifically, R¹ is methyl.

Specifically, R² is 1,3,4-thiadiazol-2-yl.

Specifically, R² is 3-methoxy-1,2,4-thiadiazol-5-yl.

In another aspect, the present invention also provides:

pharmaceutical compositions which comprise a pharmaceutically acceptable carrier and a compound of formula I,

methods for controlling or treating a disorder or condition selected from organ transplant rejections, lupus, multiple sclerosis, rheumatoid arthritis, psoriasis, cancer, osteoarthritis, and diabetes by administering to a mammal in need a therapeutically effective amount of a compound of formula I or a pharmaceutically acceptable salt thereof,

methods for controlling or treating a disorder or condition selected from diabetes, cancer, autoimmune thyroid disorders, ulcerative colitis, Crohn's disease, dry eyes, Alzheimer's disease, leukemia, and other indications where immunosuppression or immunomodulation would be desirable by administering to a mammal in need a therapeutically effective amount of a compound of formula I or a pharmaceutically acceptable salt thereof,

methods for controlling or treating a disorder or condition selected from allergic reaction including allergic dermatitis, eczema, atopic dermatitis, pruritus and other pruritic conditions and inflammatory disease such as bowel disease in mammal by administering to a mammal in need a therapeutically effective amount of a compound of formula I or a pharmaceutically acceptable salt thereof,

methods for controlling or treating a disorder or condition selected from Asthma and other obstructive airways diseases, including chronic or inveterate asthma, late asthma, airway hyper-responsiveness, bronchitis, bronchial asthma, allergic asthma, intrinsic asthma, extrinsic asthma, dust asthma, recurrent airway obstruction, and chronic obstruction pulmonary disease by administering to a mammal in need a therapeutically effective amount of a compound of formula I or a pharmaceutically acceptable salt thereof,

methods for the inhibition of protein tyrosine kinases or JAK-1, JAK-2, JAK-3 and/or Tyk-2 by administering to a mammal in need of a therapeutically effective amount of a compound of formula I or a pharmaceutically acceptable salt thereof, and

methods for the preparation of compounds of the present invention.

### DETAILED DESCRIPTION

With respect to the above compound, and throughout the application and claims, the following terms have the meanings defined below.

The term “mammal” refers to human or animals including livestock and companion animals. The phrase “companion animal” or “companion animals” refers to animals kept as pets. Examples of companion animals include cats, dogs, and horses. The term “livestock” refers to animals reared or raised in an agricultural setting to make products such as food or fiber, or for its labor. In some embodiments, livestock are suitable for consumption by mammals, for example humans. Examples of livestock animals include mammals, such as cattle, goats, horses, pigs, sheep, including lambs, and rabbits, as well as birds, such as chickens, ducks and turkeys.

The term “controlling”, “treating” or “treatment” of a disease includes: (1) preventing the disease, i.e. causing the clinical symptoms or signs of the disease not to develop in a mammal that may be exposed to or predisposed to the disease but does not yet experience or display symptoms/signs of the disease; (2) inhibiting the disease, i.e., arresting or reducing the development of the disease or its clinical symptoms/signs; or (3) relieving the disease, i.e., causing regression of the disease or its clinical symptoms/signs.

The term “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

The term “pharmaceutically acceptable” means suitable for use in mammals, companion animals or livestock animals.

The carbon atom content of various hydrocarbon-containing moieties is indicated by a prefix designating the minimum and maximum number of carbon atoms in the moiety, i.e., the prefix Cᵢ₋ⱼ indicates a moiety of the integer “i” to the integer “j” carbon atoms, inclusive. Thus, for example, C₁₋₄ alkyl refers to alkyl of one to four carbon atoms, inclusive.

The term alkyl refers to straight, branched and a cyclic saturated monovalent hydrocarbon groups, but reference to an individual radical such as “propyl” embraces only the straight chain radical, a branched chain isomer such as “isopropyl” or a cyclic isomer such as cyclopropylmethyl or cyclopentyl being specifically referred to.

Compounds that have the same molecular formula but differ in the nature or sequence of bonding of their atoms or the arrangement of their atoms in space are termed “isomers”. Isomers that differ in the arrangement of their atoms in space are termed “stereoisomers”. It will be appreciated by those skilled in the art that the compound of formula I can exist as cis- and trans-achiral diastereomers.

Included within the scope of the described compounds are all isomers (e.g. cis-, trans-, or diastereomers) of the compounds described herein alone as well as any mixtures. All of these forms, including enantiomers, diastereomers, cis, trans, syn, anti, solvates (including hydrates), tautomers, and mixtures thereof, are included in the described compounds.

Specifically, the present invention provides a compound of formula IA,

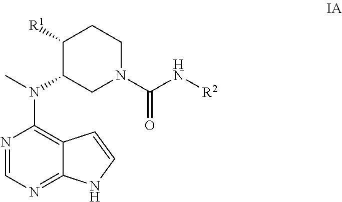

or a pharmaceutically acceptable salt thereof.

Specifically, R¹ is methyl.

Specifically, R² is 1,3,4-thiadiazol-2-yl, or 1,2,4-thiadiazol-5-yl.

Specifically, R² is 3-methoxy-1,2,4-thiadiazol-5-yl.

Specifically, a compound of formula IA is (3R,4R)-4-methyl-3-[methyl(7H-pyrrolo[2,3-d]pyrimidin-4-yl)amino]-N-(1,3,4-thiadiazol-2-yl)piperidine-1-carboxamide or (3R,4R)-N-(3-methoxy-1,2,4-thiadiazol-5-yl)-4-methyl-3-[methyl(7H-pyrrolo[2,3-d]pyrimidin-4-yl)amino]piperidine-1-carboxamide.

Stereoisomeric mixtures, e.g. mixtures of diastereomers, can be separated into their corresponding isomers in a known manner by means of suitable separation methods. Diastereomeric mixtures for example may be separated into their individual diastereomers by means of fractionated crystallization, chromatography, solvent distribution, and similar procedures. This separation may take place either at the level of one of the starting compounds or in a compound of formula I itself Enantiomers may be separated through the formation of diastereomeric salts, for example by salt formation with an enantiomer-pure chiral acid, or by means of chromatography, for example by HPLC, using chromatographic substrates with chiral ligands.

### Routes of Administration

In therapeutic use for treating disorders in a mammal (i.e. human and animals), a compound of the present invention or its pharmaceutical compositions can be administered orally, parenterally, topically, rectally, transmucosally, or intestinally. Parenteral administrations include indirect injections to generate a systemic effect or direct injections to the afflicted area. Topical administrations include the treatment of skin or organs readily accessibly by local application, for example, eyes or ears. It also includes transdermal delivery to generate a systemic effect. The rectal administration includes the form of suppositories. The preferred routes of administration are oral and parenteral.

### Pharmaceutical Salts

The compound of formula I may be used in its native form or as a salt. In cases where forming a stable nontoxic acid or base salt is desired, administration of the compound as a pharmaceutically acceptable salt may be appropriate. Pharmaceutically acceptable salts of the compounds of formula I include the acetate, ascorbate, aspartate, benzoate, besylate, bicarbonate/carbonate, bisulphate/sulphate, borate, camsylate, citrate, edisylate, etoglutarate, esylate, formate, fumarate, gluceptate, gluconate, glucuronate, glycerophosphate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

### Composition/Formulation

Pharmaceutical compositions of the present invention may be manufactured by processes well known in the art, e.g., by means of conventional mixing, dissolving, granulation, dragee-making, levigating, emulsifying, encapsulating, entrapping, lyophilizing processes or spray drying.

Pharmaceutical compositions for use in accordance with the present invention may be formulated in conventional manner using one or more pharmaceutically acceptable carriers comprising excipients and auxiliaries, which facilitate processing of the active compound into preparations, which can be used pharmaceutically. Proper formulation is dependent upon the route of administration chosen. Pharmaceutically acceptable excipients and carriers are generally known to those skilled in the art and are thus included in the instant invention. Such excipients and carriers are described, for example, in “Remington's Pharmaceutical Sciences” Mack Pub. Co., New Jersey (1991).

The formulations of the invention can be designed to be short-acting, fast-releasing, long-acting, and sustained-releasing. Thus, the pharmaceutical formulations can also be formulated for controlled release or for slow release.

### Dosage

Pharmaceutical compositions suitable for use in the present invention include compositions wherein the active ingredients are contained in an amount sufficient to achieve the intended purpose, i.e., control or the treatment of disorders or diseases. More specifically, a therapeutically effective amount means an amount of compound effective to prevent, alleviate or ameliorate symptoms/signs of disease or prolong the survival of the subject being treated.

The quantity of active component, which is the compound of this invention, in the pharmaceutical composition and unit dosage form thereof, may be varied or adjusted widely depending upon the manner of administration, the potency of the particular compound and the desired concentration. Determination of a therapeutically effective amount is well within the capability of those skilled in the art. Generally, the quantity of active component will range between 0.01% to 99% by weight of the composition.

Generally, a therapeutically effective amount of dosage of active component will be in the range of about 0.01 to about 100 mg/kg of body weight/day, preferably about 0.1 to about 10 mg/kg of body weight/day, more preferably about 0.2 to 3 mg/kg of body weight/day, even more preferably about 0.2 to 1.5 mg/kg of body weight/day. It is to be understood that the dosages may vary depending upon the requirements of each subject and the severity of the disorders or diseases being treated.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Also, it is to be understood that the initial dosage administered may be increased beyond the above upper level in order to rapidly achieve the desired plasma concentration. On the other hand, the initial dosage may be smaller than the optimum and the daily dosage may be progressively increased during the course of treatment depending on the particular situation. If desired, the daily dose may also be divided into multiple doses for administration, e.g., two to four times per day.

### Medical and Veterinary Uses

Compounds of the present invention are Janus Kinase inhibitors (JAK-i) with efficacy against Janus Kinase-1 (JAK-1), Janus Kinase-2 (JAK-2) and Janus Kinase-3 (JAK-3). Accordingly, they are useful as therapeutic agents for organ transplants, lupus, multiple sclerosis, rheumatoid arthritis, psoriasis, Type I diabetes and complications from diabetes, cancer, asthma, atopic dermatitis, autoimmune thyroid disorders, ulcerative colitis, Crohn's disease, Alzheimer's disease, leukemia, osteoarthritis, myeloproliferative diseases such as polycythemia Vera (PV) and essential thrombocythemia (ET), control of pruritus, chronic respiratory disease and other indications where immunosuppression/immunomodulation would be desirable.

In addition, there are substantial needs for safe and efficacious agents to control atopic dermatitis in animals. The market for treating atopic dermatitis in animals is currently dominated by corticosteroids, which cause distressing and undesirable side effects in animals, specifically in companion animals such as dogs. Antihistamines are also used, but are poorly effective. A canine formulation of cyclosporine (ATOPICA™) is currently being marketed for atopic dermatitis, but is expensive and has a slow onset of efficacy. In addition, there are GI toleration issues with ATOPICA™. Compounds of present invention are JAK inhibitors with efficacy against JAK-1 and JAK-3. These compounds will be an alternative to steroid usage and provide resolution of chronic pruritus and inflammation that would either persist in atopic dermatitis or slowly regress following removal of allergen or causative agent, such as fleas in flea-allergic dermatitis.

Compounds of the present invention may be administered in a pharmaceutically acceptable form either alone or in combination with one or more additional agents which modulate a mammalian immune system or with antiinflammatory agents. These agents may include but are not limited to cyclosporin A (e.g. Sandimmune® or Neoral®, rapamycin, FK-506 (tacrolimus), leflunomide, deoxyspergualin, mycophenolate (e.g. Cellcept®, azathioprine (e.g. Imuran®), daclizumab (e.g. Zenapax®), OKT3 (e.g. Orthocolone®), AtGam, aspirin, acetaminophen, ibuprofen, naproxen, piroxicam, and antiinflammatory steroids (e.g. prednisolone or dexamethasone). These agents may be administered as part of the same or separate dosage forms, via the same or different routes of administration, and on the same or different administration schedules according to standard pharmaceutical practice known to one skilled in the art.

In one embodiment, the invention provides methods of treating or preventing a disease, condition or disorder associated with JAK in a subject, such as a human or non-human mammal, comprising administering an effective amount of one or more compounds described herein to the subject. The JAK associated disease, condition or disorder can be related to JAK-1, JAK-2, JAK-3, and/or Tyk-2. Suitable subjects that can be treated include domestic or wild animals, companion animals, such as dogs, cats, horses and the like; livestock including, cows and other ruminants, pigs, poultry, rabbits and the like; primates, for example monkeys, such as rhesus monkeys and cynomolgus (also known as crab-eating or long-tailed) monkeys, marmosets, tamarins, chimpanzees, macaques and the like; and rodents, such as rats, mice, gerbils, guinea pigs and the like. In one embodiment, the compound is administered in a pharmaceutically acceptable form, optionally in a pharmaceutically acceptable carrier.

JAK/STAT signaling has been implicated in the mediation of many abnormal immune responses such as allergies, asthma, autoimmune diseases such as transplant (allograft) rejection, rheumatoid arthritis, amyotrophic lateral sclerosis and multiple sclerosis, as well as in solid and hematologic malignancies such as leukemia and lymphomas. For a review of the pharmaceutical intervention of the JAK/STAT pathway see Frank, (1999), Mol. Med. 5:432:456 and Seidel et al., (2000), Oncogene 19:2645-2656.

JAK-3 in particular has been implicated in a variety of biological processes. For example, the proliferation and survival of murine mast cells induced by IL-4 and IL-9 have been shown to be dependent on JAK-3 and gamma chain-signaling. Suzuki et al., (2000), Blood 96:2172-2180. JAK-3 also plays a crucial role in IgE receptor-mediated mast cell degranulation responses (Malaviya et al., (1999), Biochem. Biophys. Res. Commun 257:807-813), and inhibition of JAK-3 kinase has been shown to prevent type I hypersensitivity reactions, including anaphylaxis (Malaviya et al., (1999), J. Biol. Chem. 274:27028-27038). JAK-3 inhibition has also been shown to result in immune suppression for allograft rejection (Kirken, (2001), Transpl. Proc. 33:3268-3270). JAK-3 kinases have also been implicated in the mechanism involved in early and late stages of rheumatoid arthritis (Muller-Ladner et al., (2000), J. Immunol. 164:3894-3901); familial amyotrophic lateral sclerosis (Trieu et al., (2000), Biochem Biophys. Res. Commun 267:22-25); leukemia (Sudbeck et al., (1999), Clin. Cancer Res. 5:1569-1582); mycosis fungoides, a form of T-cell lymphoma (Nielsen et al., (1997), Proc. Natl. Acad. Sci. USA 94:6764-6769); and abnormal cell growth (Yu et al., (1997), J. Immunol. 159:5206-5210; Catlett-Falcone et al., (1999), Immunity 10:105-115).

The JAK kinases, including JAK-1 and JAK-3, are abundantly expressed in primary leukemic cells from children with acute lymphoblastic leukemia, the most common form of childhood cancer, and studies have correlated STAT activation in certain cells with signals regulating apoptosis (Demoulin et al., (1996), Mol. Cell. Biol. 16:4710-6; Jurlander et al., (1997), Blood 89:4146-52; Kaneko et al., (1997), Clin. Exp. Immun. 109:185-193; and Nakamura et al., (1996), J. Biol. Chem. 271: 19483-8). They are also known to be important to lymphocyte differentiation, function and survival. JAK-3 in particular plays an essential role in the function of lymphocytes, macrophages, and mast cells. Given the importance of this JAK kinase, compounds which modulate the JAK pathway, including those selective for JAK-1 and JAK-3, can be useful for treating diseases or conditions where the function of lymphocytes, macrophages, or mast cells is involved (Kudlacz et al., (2004) Am. J. Transplant 4:51-57; Changelian (2003) Science 302:875-878).

Conditions in which targeting of the JAK pathway or modulation of the JAK kinases, particularly JAK-1 and JAK-3, are contemplated to be therapeutically useful include, arthritis, asthma, autoimmune diseases, cancers or tumors, diabetes, certain eye diseases, disorders or conditions, inflammation, intestinal inflammations, allergies or conditions, neurodegenerative diseases, psoriasis, transplant rejection, and viral infection. Conditions which can benefit for inhibition of JAK-1 and JAK-3 are discussed in greater detail below.

Accordingly, the compound of formula I or its pharmaceutically acceptable salts and pharmaceutical compositions can be used to treat a variety of conditions or diseases such as:

Arthritis, including rheumatoid arthritis, juvenile arthritis, and psoriatic arthritis;

Asthma and other obstructive airways diseases, including chronic or inveterate asthma, late asthma, airway hyper-responsiveness, bronchitis, bronchial asthma, allergic asthma, intrinsic asthma, extrinsic asthma, dust asthma, recurrent airway obstruction, and chronic obstruction pulmonary disease;

Autoimmune diseases or disorders, including those designated as single organ or single cell-type autoimmune disorders, for example Hashimoto's thyroiditis, autoimmune hemolytic anemia, autoimmune atrophic gastritis of pernicious anemia, autoimmune encephalomyelitis, autoimmune orchitis, Goodpasture's disease, autoimmune thrombocytopenia, sympathetic ophthalmia, myasthenia gravis, Graves' disease, primary biliary cirrhosis, chronic aggressive hepatitis, ulcerative colitis and membranous glomerulopathy, those designated as involving systemic autoimmune disorder, for example systemic lupus erythematosis, rheumatoid arthritis, Sjogren's syndrome, Reiter's syndrome, polymyositis-dermatomyositis, systemic sclerosis, polyarteritis nodosa, multiple sclerosis and bullous pemphigoid, and additional autoimmune diseases, which can be O-cell (humoral) based or T-cell based, including Cogan's syndrome, ankylosing spondylitis, Wegener's granulomatosis, autoimmune alopecia, Type I or juvenile onset diabetes, and thyroiditis;

Cancers or tumors, including alimentary/gastrointestinal tract cancer, colon cancer, liver cancer, skin cancer including mast cell tumor and squamous cell carcinoma, breast and mammary cancer, ovarian cancer, prostate cancer, lymphoma, leukemia, including acute myelogenous leukemia and chronic myelogenous leukemia, kidney cancer, lung cancer, muscle cancer, bone cancer, bladder cancer, brain cancer, melanoma including oral and metastatic melanoma, Kaposi's sarcoma, myelomas including multiple myeloma, myeloproliferative disorders, proliferative diabetic retinopathy, and angiogenic-associated disorders including solid tumors;

Diabetes, including Type I diabetes and complications from diabetes;

Eye diseases, disorders or conditions including autoimmune diseases of the eye, keratoconjunctivitis, vernal conjunctivitis, uveitis including uveitis associated with Behcet's disease and lens-induced uveitis, keratitis, herpetic keratitis, conical keratitis, corneal epithelial dystrophy, keratoleukoma, ocular premphigus, Mooren's ulcer, scleritis, Grave's ophthalmopathy, Vogt-Koyanagi-Harada syndrome, keratoconjunctivitis sicca (dry eye), phlyctenule, iridocyclitis, sarcoidosis, endocrine ophthalmopathy, sympathetic ophthalmitis, allergic conjunctivitis, and ocular neovascularization;

Intestinal inflammations, allergies or conditions including Crohn's disease and/or ulcerative colitis, inflammatory bowel disease, coeliac diseases, proctitis, eosinophilic gastroenteritis, and mastocytosis;

Neurodegenerative diseases including motor neuron disease, Alzheimer's disease, Parkinson's disease, amyotrophic lateral sclerosis, Huntington's disease, cerebral ischemia, or neurodegenerative disease caused by traumatic injury, strike, glutamate neurotoxicity or hypoxia; ischemic/reperfusion injury in stroke, myocardial ischemia, renal ischemia, heart attacks, cardiac hypertrophy, atherosclerosis and arteriosclerosis, organ hypoxia, and platelet aggregation;

Skin diseases, conditions or disorders including atopic dermatitis, eczema, psoriasis, scleroderma, pruritus and other pruritic conditions;

Allergic reactions including allergic dermatitis in mammal including horse allergic diseases such as bite hypersensitivity, summer eczema and sweet itch in horses; and

Transplant rejection, including pancreas islet transplant rejection, bone marrow transplant rejection, graft-versus-host disease, organ and cell transplant rejection such as bone marrow, cartilage, cornea, heart, intervertebral disc, islet, kidney, limb, liver, lung, muscle, myoblast, nerve, pancreas, skin, small intestine, or trachea, and xeno transplantation.

Another embodiment provides a method of inhibiting a JAK enzyme, including JAK-1, JAK-2, JAK-3 and/or Tyk-2, that includes contacting the JAK enzyme with either a non-therapeutic amount or a therapeutically effective amount of one or more of the present compounds. Such methods can occur in vivo or in vitro. In vitro contact can involve a screening assay to determine the efficacy of the one or more compounds against a selected enzyme at various amounts or concentrations. In vivo contact with a therapeutically effective amount of the one or more compounds can involve treatment of a described disease, disorder or condition or prophylaxis of organ transplant rejection in the animal in which the contact occurs. The effect of the one or more compounds on the JAK enzyme and/or host animal can also be determined or measured. Methods for determining JAK activity include those described in the Examples as well as those disclosed in WO 99/65908, WO 99/65909, WO 01/42246, WO 02/00661, WO 02/096909, WO 2004/046112 or WO 2007/012953.

The following reaction schemes illustrate the general synthetic procedures of the compounds of the present invention. All starting materials are prepared by procedures described in these schemes or by procedures known to one of ordinary skill in the art.

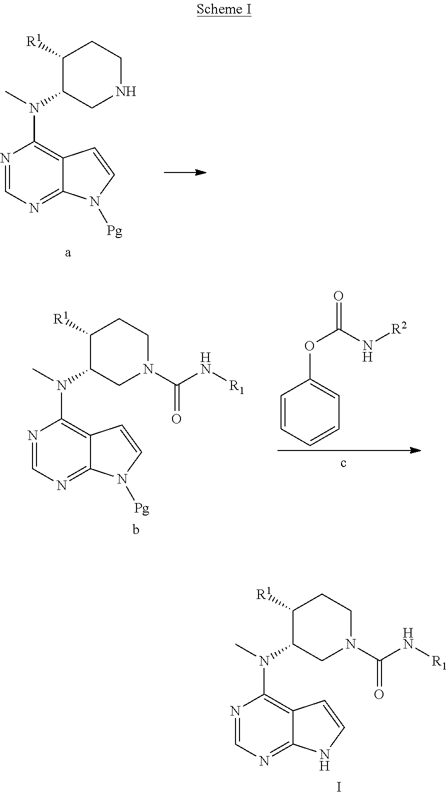

It will be apparent to those skilled in the art that sensitive functional groups (Pg) may need to be protected and deprotected during synthesis of a compound of the invention. This may be achieved by conventional methods, for example as described in “Protective Groups in Organic Synthesis” by T W Greene and P G M Wuts, John Wiley & Sons Inc (1999), and references therein.

In Scheme I, a compound of structure (b) can be obtained from a compound of structure (a) according to the procedures described in WO200701295) by reaction with a suitable carbamate such as a phenyl thiadiazole carbamate group using procedures well known in the art.

Compounds of formula I of the present invention may be prepared from compounds of formula (b) wherein Pg is a suitable protecting group by deprotection procedures known to one skilled in the art. For example, when the protecting group (Pg) is tosyl, suitable deprotection conditions involve reaction with a base such as lithium hydroxide or cesium carbonate in a protic solvent such as methanol or isopropanol and optionally miscible cosolvents such as tetrahydrofuran and water at room temperature for several hours, to produce the deprotected amine of formula I.

### EXAMPLES

### Example 1

### Preparation of (3R,4R)-4-methyl-3-[methyl(7H-pyrrolo[2,3-d]pyrimidin-4-yl)amino]-N-(1,3,4-thiadiazol-2-yl)piperidine-1-carboxamide

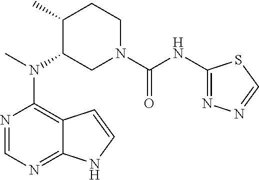

### Step 1: Preparation of (3R,4R)-4-methyl-3-(methyl{7-[(4-methylphenyl)sulfonyl]-7H-pyrrolo[2,3-d]pyrimidin-4-yl}amino)-N-(1,3,4-thiadiazol-2-yl)piperidine-1-carboxamide

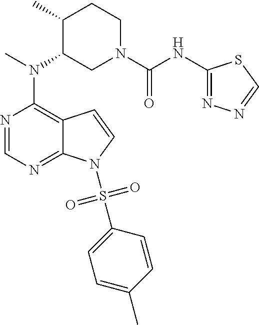

N-methyl-7-[(4-methylphenyl)sulfonyl]-N-[(3R,4R)-4-methylpiperidin-3-yl]-7H-pyrrolo[2,3-d]pyrimidin-4-amine (prepared according to the procedures described in WO2007012953) is dissolved in THF (5 mL) and phenyl 1,3,4-thiadiazol-2-ylcarbamate (196 mg, 0.89 mmol) along with triethylamine (0.19 mL, 1.3 mmol). The mixture is heated to 60° C. for about three hours then at room temperature overnight. The solvent is removed under reduced pressure and the crude compound purified by flash column chromatography on silica gel eluting from neat DCM to 10% MeOH/DCM to give the title compound as white solid.

### m/z (CI) 527 ([M+H]⁺.

### Step 2: Preparation of (3R,4R)-4-methyl-3-[methyl(7H-pyrrolo[2,3-d]pyrimidin-4-yl)amino]-N-(1,3,4-thiadiazol-2-yl)piperidine-1-carboxamide

The compound from Preparation 1 (170 mg) is dissolved in methanol (5 mL) and lithium hydroxide (8 mg) is added. The reaction mixture is heated to about 60° C. for about two hours. The solvent is removed under reduced pressure and the resultant residue is purified by flash column chromatography on amino supported silica gel to give the title compound as a white solid.

¹H NMR (DMSO-d₆): 11.65 (1H), 11.44 (1H), 8.96 (1H), 8.12 (1H), 7.15-7.14 (1H), 6.58 (1H), 4.91 (1H), 4.02-3.77 (3H), 3.55-3.49 (1H), 3.34 (3H), 2.43-2.40 (1H), 1.82-1.76 (1H), 1.64-1.60 (1H), 1.06-1.04 (3H); m/z (CI) 373 ([M+H]⁺.

### Example 2

### Preparation of (3R,4R)-N-(3-methoxy-1,2,4-thiadiazol-5-yl)-4-methyl-3-[methyl(7H-pyrrolo[2,3-d]pyrimidin-4-yl)amino]piperidine-1-carboxamide

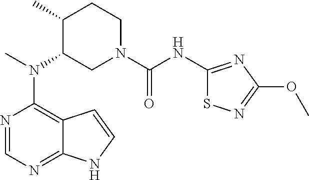

Triethylamine is added to a solution of 5-Amino-3-methoxy-1,2,4-thiadiazole and phosgene (0.72 mL, 1.22 mmol, 1.7M) in DCM (6 mL) at 0° C. After about 15 minutes N-methyl-N-[(3R,4R)-4-methylpiperidin-3-yl]-7H-pyrrolo[2,3-d]pyrimidin-4-amine (250 mg, 1.02 mmol) is added. After stirring for about 1 hour the mixture is partitioned between water (50 mL) and DCM (50 mL). The organics are separated, dried over MgSO₄, filtered and evaporated to give a yellow oil, which is further purified by column chromatography eluting from neat DCM to 10% MeOH/DCM to give the title compound as a white solid.

### m/z (CI) 403 ([M+H]⁺.

### Example 3

### Preparation of (3R,4R)-N-(4-cyano-3-methylisoxazol-5-yl)-4-methyl-3-[methyl(7H-pyrrolo[2,3-d]pyrimidin-4-yl)amino]piperidine-1-carboxamide

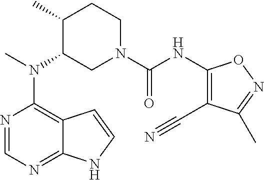

Following the general procedure of Example 1, and making non-critical variations but using phenyl 4-cyano-3-methylisoxazol-5-yl carbamate as the starting material, the title compound is obtained.

### Example 4

### Preparation of (3R,4R)-4-methyl-N-[1-methyl-3-(methylsulfanyl)-1H-1,2,4-triazol-5-yl]-3-[methyl(7H-pyrrolo[2,3-d]pyrimidin-4-yl)amino]piperidine-1-carboxamide

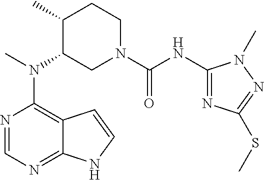

Following the general procedure of Example 1, and making non-critical variations but using phenyl)-1H-1,2,4-triazol-5-yl carbamate as the starting material, the title compound is obtained.

### Example 5

### Canine in vitro T-cell Proliferation Assay

Introduction: T-cell activation plays a key role in a variety of inflammatory and autoimmune disorders as well as asthma, allergies and pruritus. Since T-cell activation can, in part, can be triggered by cytokines that signal through the JAK-STAT pathway, a JAK inhibitor could be effective against such diseases involving aberrant T-cell activation. Compounds inhibiting T-cell proliferation in the low nM range suggest effectiveness against inhibiting JAK1 and JAK3 enzyme in cells.
Methods: Canine whole blood is collected in heparin sulfate tubes from beagle dogs. Whole blood (20 μL) is plated in 96-well plates (Costar) with 180 μL of DMEM complete medium (Dublecco's Modified Eagle Medium; 10% heat inactivated fetal bovine serum; 100 u/ml penicillin; 100 ug/mL streptomycin, from Gibco) containing vehicle control or test compound (0.001 to 10 μM), concanavalin A (ConA; 1 μg/ml), and canine interleukin-2 (IL-2; 50 ng/ml). Wells containing whole blood, medium with vehicle control and no ConA or IL-2 are used as background (BKG) controls. Plates are incubated at 37° C. for 48 hrs. Tritiated thymidine, 0.4 μCi/well (Perkin Elmer), is added for 20 additional hours. Plates are frozen and then thawed, washed and filtered using a Brandel MLR-96 cell harvester and prewet filter mats (Wallac 1205-401, Perkin Elmer). Filters are dried at 60° C. for one hour (Precision 16EG convection oven) and placed into filter sample bags (Wallac 1205-411, Perkin Elmer) with 10 mL of scintillant (Wallac 1205-440, Perkin Elmer). Sealed filters are counted on a LKB Wallac 1205 Betaplate liquid scintillation counter. Data are collected via Gterm Betaplate program v1.1 (Wallac copyright 1989-1990) and transformed into percent inhibition, calculated using the following formula:

$$100 - \frac{( \begin{matrix}Mean Compound Treatment cpm - \\ Mean BKG cpm\end{matrix} )}{( \begin{matrix}Mean Vehicle control treatment cpm - \\ Mean BKG cpm\end{matrix} )} \times 100 = \% Inhibition$$

Data are graphically displayed as percent inhibition using GraphPad Prism 4.00, and IC IC₅₀ curves are fitted using a point to point analysis. The average assay results for Examples 1-4 are shown in Table 1.

TABLE 1
Canine in vitro T-cell Proliferation Assay

| Example No. | IC₅₀ (nM) |
|---|---|
| 1 | 28.5 |
| 2 | 50 |
| 3 | >10,000 |
| 4 | >10,000 |

1. A compound of formula I: 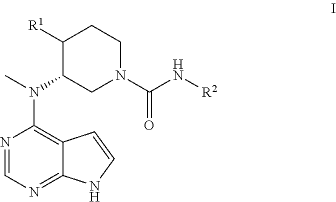
or a pharmaceutically acceptable salt thereof wherein R¹ is H or —C₁₋₄alkyl; and R² is a thiadiazole group optionally substituted with —OH or —OCH₃.

2. A compound of claim 1 which is a compound of formula IA 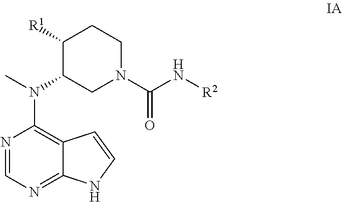
or a pharmaceutically acceptable salt thereof.

3. A compound of claim 1 wherein R¹ is methyl.

4. A compound of claim 1 wherein R² is 1,3,4-thiadiazol-2-yl.

5. A compound of claim 1 wherein R² is 3-methoxy-1,2,4-thiadiazol-5-yl.

6. A compound of claim 1 which is (3R,4R)-4-methyl-3-[methyl(7H-pyrrolo[2,3-d]pyrimidin-4-yl)amino]-N-(1,3,4-thiadiazol-2-yl)piperidine-1-carboxamide.

7. A compound of claim 1 which is (3R,4R)-N-(3-methoxy-1,2,4-thiadiazol-5-yl)-4-methyl-3-[methyl(7H-pyrrolo[2,3-d]pyrimidin-4-yl)amino]piperidine-1-carboxamide.

8. A pharmaceutical composition comprising a compound of claim 1, or a pharmaceutically acceptable salt thereof and a pharmaceutically acceptable carrier.

9. A method for treating allergic reactions, allergic dermatitis, atopic dermatitis, eczema, or pruritus in a mammal comprising administering to a mammal in need a therapeutically effective amount of a compound of claim 1.

10. The method of claim 9 wherein the therapeutically effective amount is from 0.01 mg/kg of body weight/day to 100 mg/kg of body weight/day.

11. The method of claim 9 wherein the therapeutically effective amount is from 0.1 mg/kg of body weight/day to 10 mg/kg of body weight/day.

12. The method of claim 9 wherein the therapeutically effective amount is from 0.2 mg/kg of body weight/day to 1.5 mg/kg of body weight/day.

13. The method of claim 9 wherein the mammal comprises a companion animals.

14. The method of claim 3 wherein the companion animals is a dog.

15. The method of claim 9 wherein the mammal comprises livestock.

16. The method of claim 9 wherein the compound of formula 1 is administered orally, parenterally, or topically.

17. A method for treating neurodegenerative diseases, keratoconjunctivitis, chronic respiratory diseases, autoimmune diseases, inflammatory bowel disease, neoplasia and arthritic conditions in a mammal comprising administering to a mammal in need a therapeutically effective amount of a compound of claim 1.
